(19) United States
(12) Patent Application Publication
(10) Pub. No.: US 2022/0064454 A1
(43) Pub. Date: Mar. 3, 2022

(54) METHODS FOR EXTRACTION OF MELANIN FROM FUNGAL SPECIES

(71) Applicant: Birch Boys, Inc., Tupper Lake, NY (US)

(72) Inventors: Garrett Kopp, Tupper Lake, NY (US); Maya R. Duncan-White, Souderton, PA (US)

(21) Appl. No.: 17/002,265

(22) Filed: Aug. 25, 2020

(51) Int. Cl. C09B 67/54; C09B 61/00; C12N 1/14

(52) U.S. Cl. CPC C09B 67/0096; C12N 1/14; C09B 61/00

(57) ABSTRACT

The present application discloses methods for extracting melanin from a fungus, where the fungus is Inonotus obliquus (chaga), Fomes fomentarius, Ganoderma tsugae, or Phellinus igniarius. In the methods, a sample of the fungus is dried and pulverized to form a powder sample. The powder sample is refluxed in a sodium hydroxide solution to form a mixture. The mixture is separated into a liquid portion and a solid portion via centrifugation or vacuum filtration, resulting in the liquid portion including the melanin extract. The separated liquid portion including the melanin is acidified with a hydrochloric acid solution to precipitate the melanin. The precipitated melanin is then isolated via centrifugation or vacuum filtration such that the melanin collects as the pellet or precipitate. The melanin precipitate is then purified by washing with dilute HCl and deionized, distilled water. The purified melanin is then dried to produce the extracted fungal melanin.

### TECHNICAL FIELD

The present disclosure is generally related to methods for extracting melanin from fungi samples.

### BACKGROUND OF THE DISCLOSURE

Melanin is a general term used to describe certain pigments that are found within most organisms. There are three primary types of human melanin, eumelanin (brownish-black pigment; most common), pheomelanin (yellowish-red pigment) and neuromelanin (dark brown pigment). Fungal melanin, on the other hand, can been classified according to their precursors as 1,8-dihydroxynapthalene (DHN-melanin), L-dihydroxyphenylalanine (DOPA-melanin), and glutaminyl-3,4-dihydroxybenzene (GDHB-melanin). Melanins are generally resistant to chemical degradation by acids and are insoluble in most substances. Melanins are currently used in several applications, including skincare (e.g., sunscreens, lotions) and metal chelation processes, and are being researched for further applications in the medical field.

Some fungi can be rich in melanin, and these melanized fungi have developed evolutionary advantages, including resistance to extreme temperatures, resistance to damage from ultraviolet light and oxidation, and improved mechanical strength. While methods exist for extracting melanin from fungi, there are several barriers to successful extraction. For example, most of the melanin in fungi is located near or in the cell wall and can be associated with chitin. As such, the melanin must be separated from the cell wall and disassociated from chitin during the extraction process. Previous extraction methods have used enzymes to isolate the melanin; however, enzymes for such a process can be very costly. Similarly, previous extraction methods have implemented dialysis steps, which are also costly and time-intensive. Further, previous extraction methods have often produced melanin samples with high levels of impurities, which then require further processing for use in downstream applications such as sunscreens and lotions.

Chaga mushrooms (Inonotus obliquus) exhibit several beneficial attributes, including strong antioxidant activity, and thus is a candidate for further research, including melanin extraction. However, similar to other fungi species, most melanin granules in chaga are localized in the cell wall and are cross-linked to polysaccharides or chitin. Despite the emerging interest in chaga, few methods are known for efficiently extracting melanin from chaga. The present application addresses these and other challenges related to the extraction of melanin from fungi species, including Inonotus obliquus.

### SUMMARY OF THE DISCLOSURE

In a first aspect, a method for extracting melanin from a fungal species is provided. In the method, a sample of fungus is dried and pulverized, where the sample of fungus is selected from the group consisting of Inonotus obliquus, Fomes fomentarius, Ganoderma tsugae, and Phellinus igniarius. The sample is refluxed in a sodium hydroxide solution to form a mixture, and the mixture is then separated into a liquid portion and a solid portion via centrifugation or vacuum filtration. The centrifugation or vacuum filtration results in the liquid portion comprising melanin. The liquid portion is separated from the solid portion and then acidified to a pH of approximately 2 or less than 2 with a hydrochloric acid solution to precipitate melanin. The precipitated melanin is then isolated via centrifugation or vacuum filtration. The isolated melanin is then purified with at least one of HCl and deionized, distilled water. The purified melanin is then dried.

In another aspect, at least one melanin confirmation test is performed on the purified melanin. The at least one test is selected from the group consisting of: confirming that the purified melanin is black-dark brown, confirming that the purified melanin is soluble in an alkaline solution, confirming that the purified melanin is insoluble in hot and cold concentrated acids, and confirming that a reaction of the purified melanin with an oxidizing agent results in bleaching of the purified melanin.

In another aspect, the NaOH solution has a molar concentration in a range of approximately 0.5M-2M. In a further aspect, the NaOH solution is 1M NaOH and the pulverized sample is combined with the 1M NaOH in a container at a ratio of approximately 0.01-0.1 g/mL.

In another aspect, the sample is refluxed for 1-5 hours at a temperature in the range of approximately 100° C.-103° C. In a further aspect, the sample is refluxed for approximately 2 hours in a container and the container is immersed in a mineral oil bath maintained at approximately 100° C.

In another aspect, the step of separating the mixture via centrifugation or vacuum filtration comprises centrifuging the mixture at a relative centrifugal force (RCF) of approximately 4500-5000 for approximately 10-25 minutes. In another aspect, the step of separating the mixture via centrifugation or vacuum filtration comprises vacuum filtrating the sample using a vacuum pump and a 0.22-micron filtration paper.

In another aspect, the HCl solution for acidifying the separated liquid portion is approximately 10%-37% HCl solution. In another aspect, the step of isolating the precipitated melanin includes centrifugation of the precipitated melanin at approximately 4500-5000 RCF for approximately 10-25 minutes or vacuum filtration of the precipitated melanin using a vacuum pump and a 0.22-micron filtration paper.

In another aspect, the step of purifying the melanin includes centrifuging or vacuum filtrating the melanin with HCl 3 times and subsequently centrifuging or vacuum filtrating the melanin with deionized distilled water 2 times, wherein the HCl is less than 0.5M HCl. In another aspect, the step of drying the melanin includes heating the melanin at approximately 70° C. to evaporate any remaining water. In a further aspect, the heating comprises irradiating with microwaves to remove water by boiling.

In a second aspect, another method for extracting melanin from Inonotus obliquus (chaga) mushroom is provided. In the method, a sample of chaga mushroom is dried and pulverized. The sample is combined with a 1M sodium hydroxide solution in a container in a ratio of approximately 0.01-0.1 g/mL to form a mixture. The mixture is refluxed for approximately 2 hours at approximately 100° C. The mixture is then separated via centrifugation at approximately 4500 RCF for approximately 10-25 minutes to form a liquid portion that includes melanin, and a solid portion. The separated liquid portion is acidified to a pH of approximately 2 or less than 2 with a hydrochloric acid solution to precipitate melanin. The precipitated melanin is isolated via vacuum filtration. The melanin is then purified via centrifugation with at least one of HCl and deionized, distilled water. The purified melanin is then dried via irradiation with microwaves.

In another aspect, the solid portion from the separating step is recycled for further centrifugation to extract additional melanin. In another aspect, isolating the precipitated melanin includes vacuum filtration using a vacuum pump and a 0.22-micron filtration paper. In another aspect, the step of purifying the melanin includes centrifuging the melanin with HCl and subsequent centrifuging the melanin with deionized, distilled water.

In a third aspect, another method for extracting melanin from a Inonotus obliquus (chaga) mushroom is provided. In the method, a sample of chaga mushroom is dried and pulverized. The sample is then combined with a 1M sodium hydroxide solution in a container in a ratio of approximately 0.06 g/mL to form a mixture. The mixture is refluxed for approximately 2 hours at approximately 100° C. The mixture is separated via vacuum filtration to form a liquid portion comprising melanin and a solid portion. The separated liquid portion is acidified to a pH of approximately 2 or less than 2 with a hydrochloric acid solution to precipitate melanin. The precipitated melanin is isolated via centrifugation at approximately 4500 RCF. The melanin is purified via centrifugation with HCl and subsequent centrifugation with deionized, distilled water. The purified melanin is then dried via irradiation with microwaves.

In another aspect, the precipitated melanin is vacuum filtrated using a vacuum pump and a 0.22-micron filtration paper. In another aspect, the precipitated melanin is centrifuged for approximately 10-25 minutes to isolate the melanin.

### BRIEF DESCRIPTION OF THE DRAWING FIGURE

FIG. 1 shows a flow diagram of steps for a method of extracting melanin from a fungal species in accordance with one or more embodiments.

### DETAILED DESCRIPTION OF CERTAIN EMBODIMENTS

By way of overview and introduction, the present application discloses methods for extracting melanin from a fungal sample is provided. In the method, a sample of fungus is dried and pulverized, where the sample of fungus is selected from Inonotus obliquus (chaga), Fomes fomentarius, and Phellinus igniarius. In one or more embodiments, the sample is dried and pulverized to form a powder sample. The powder sample is then refluxed in a sodium hydroxide (NaOH) solution to form a mixture. The mixture is then separated into a liquid portion comprising the melanin extract and a solid portion via centrifugation or vacuum filtration. The separated liquid portion is then acidified with a hydrochloric acid (HCl) solution to precipitate the melanin. The precipitated melanin is then isolated via centrifugation or vacuum filtration, and purified with at least one of HCl and deionized, distilled water. The purified melanin is then dried, resulting in the extracted melanin substance. The melanin extracted from the fungal samples in the present method may include one or more of DHN-melanin, DOPA-melanin, and GDHB-melanin.

As further shown and described herein, the present methods provide an efficient solution for extracting melanin from a fungal sample (mushroom). The present methods also provide an improved yield of melanin from the mushroom sample and steps for removing impurities from the melanin samples. The present methods for extracting melanin from a fungal sample are now described more fully with reference to the accompanying drawings, in which one or more illustrated embodiments and/or arrangements of the methods are shown. The methods are not limited in any way to the illustrated embodiments and/or arrangements as the illustrated embodiments and/or arrangements described below are merely exemplary of the methods, which can be embodied in various forms, as appreciated by one skilled in the art. Therefore, it is to be understood that any structural and functional details disclosed herein are not to be interpreted as limiting the methods, but rather, are provided as a representative embodiment and/or arrangement for teaching one skilled in the art one or more ways to implement the methods. Furthermore, the terms and phrases used herein are not intended to be limiting, but rather are to provide an understandable description of the systems and methods. As used in the present application, the term “approximately” when used in conjunction with a number refers to any number within 10% of the referenced number, including the referenced number.

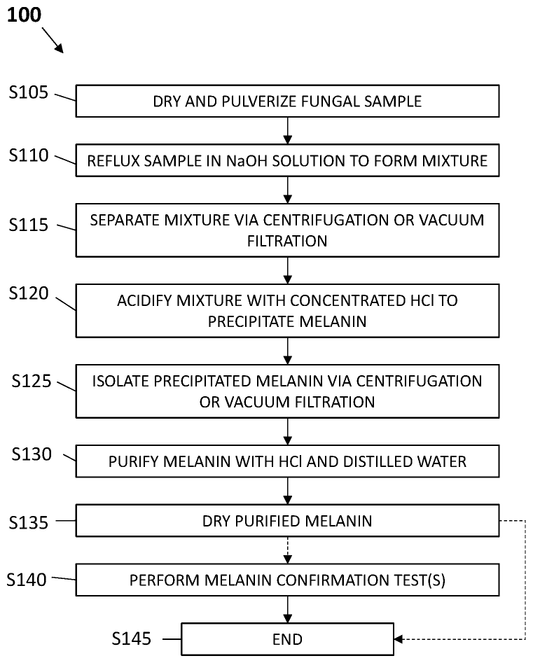

Referring now to FIG. 1, a flow diagram of steps for a method 100 of extracting melanin from a fungal sample is provided in accordance with one or more embodiments. The method 100 begins at step S105 where a sample of fungus is dried and pulverized to achieve high surface area for extraction and maximize yield. The fungus sample selected from one of the following fungal species: Inonotus obliquus (chaga), Fomes fomentarius, Ganoderma tsugae, and Phellinus igniarius. In one or more embodiments, the sample is finely pulverized into a powder form. The sample can be pulverized via any method known in the art, such as by grinding. Additionally, in one or more embodiments, the pulverized sample can be further sifted, if needed, resulted in fine particles of the sample in a powder form. In one or more embodiments, the sample can be approximately 5 grams of the fungus. In one or more embodiments in which Inonotus obliquus (chaga) is used, the sample can be collected from the exterior of the sclerotium of the fungi, or the interior of the sclerotium of the fungi, or a combination of both. More specifically, the chaga sample can be collected from an exterior of the sclerotium (black in color), or an interior of the sclerotium (orange in color), or a combination of both.

At step S110, the sample in powder form is refluxed in a NaOH solution. For refluxing, the pulverized sample is combined with the NaOH solution in a container (e.g., round bottom flask). The NaOH solution provides a highly basic environment for melanin to become soluble. In one or more embodiments, the NaOH solution is 1M NaOH. In one or more embodiments, the NaOH solution has a molar concentration in a range of approximately 0.5M-2M. In one more embodiments, the pulverized sample is combined with the NaOH solution at a ratio in the range of approximately 0.01-0.1 g/mL, and more preferably at a ratio of approximately 0.06 g/mL. The mixture of the pulverized sample and the NaOH are then refluxed over a hot plate equipped with stirring. In one or more embodiments, the mixture is refluxed for 1-5 hours at a temperature in the range of approximately 100° C.-103° C. In one or more embodiments, the mixture is refluxed for approximately 2 hours in a container and the container is immersed in a mineral oil bath maintained at approximately 100° C. In at least one embodiment stirring is maintained with a vortex for the duration the reflux step. The reflux apparatus (e.g., hot plate with stirring) can be used so that the water volume and pH is controlled and so that heat and stirring can be added to hasten the extraction process. The reflux step with NaOH extracts the melanin from the fungal cell by solubilizing it.

In at least one embodiment, prior to refluxing, the mixture of the NaOH solution and pulverized fungal sample is first sonicated with an ultrasonication device (e.g., ultrasonic bath, ultrasonic homogenizer). The mixture can be sonicated for approximately 10-20 minutes, preferably approximately 15 minutes, in order to lyse, partially lyse, or disrupt the cell walls and membranes of the fungus, thus increasing the ability of melanin to be disassociated from the cell.

At step S115, the mixture is separated into a liquid portion and a solid portion via centrifugation or vacuum filtration. Centrifugation separates components based on density, with a solid pellet collecting at the bottom of the tube (precipitate) and the extract sitting on top (supernatant). Alternatively, vacuum filtration separates solid and liquid components by pulling material through a filter paper with a defined pore size using a vacuum pump. The liquid extract will collect in the filtering flask and solids will collect on the filter paper. In one or more embodiments, the sample is centrifuged at a relative centrifugal force (RCF) or g force range of approximately 4500-5000 for approximately 10-25 minutes, more preferably 15-20 minutes, and then the supernatant (comprising the melanin extract) is pipetted out. In at least one embodiment, the sample can be centrifuged for approximately 10 minutes. In at least one embodiment, the sample is centrifuged at 4500 RCF. In one or more embodiments, the remaining solids are discarded. In at least one embodiment, the remaining solids can be recycled and centrifuged 1 or more times for extraction of additional melanin. Alternatively, in one or more embodiments in which the sample is vacuum filtrated, a vacuum pump is utilized along with filtration paper, such as a 0.22-micron filtration paper, which substantially precludes the passing of debris from fungal cells. The solids that collect on the filter paper are discarded or alternatively recycled for further vacuum filtration and extraction of melanin.

At step S120, the separated liquid portion remaining from the centrifugation or vacuum filtration, which comprises the melanin extract, is acidified with concentrated hydrochloric acid (HCl) to precipitate melanin (solid). Specifically, in one or more embodiments, the concentrated HCl is added to the liquid comprising the melanin extract in a container (e.g., beaker) to lower the pH of the liquid to approximately 2 or, alternatively, less than 2. The pH is lowered to approximately 2 or less than 2 to quickly precipitate melanin, which is insoluble in acidic solutions. In at least one embodiment, the mixture of the liquid portion and the concentrated HCl can be stirred to hasten precipitation and ensure a uniform pH. In one or more embodiments, the concentrated HCl is approximately 10-37% HCl. In one or more embodiments, the concentrated HCl is 10% HCl. The use of NaOH at step S110 and HCl at step S120 ensures that side products (e.g., NaCl) are easy to remove.

At step S125 the solid melanin precipitate is isolated via centrifugation or vacuum filtration to remove remaining liquid. Centrifugation separates solid melanin precipitate from any remaining liquid components based on density, with a solid melanin pellet collecting at the bottom of the tube and the liquid remaining on top (supernatant). The supernatant liquid can be removed using a pipette. On the other hand, vacuum filtration separates the solid melanin precipitate and liquid components by pulling material through a filter paper with defined pore size using a vacuum pump. The liquid component will collect in a container, such as a filtering flask, while the solid melanin will collect on the filter paper. In one or more embodiments, the solid melanin precipitate is centrifuged at approximately 4500-5000 RCF for approximately 15-20 minutes. In at least one embodiment, the solid melanin precipitate is centrifuged at 4500 RCF. in one or more embodiments in which the sample is vacuum filtrated, a vacuum pump and filtration paper (e.g., a 0.22-micron filtration paper) is utilized to separate solid (melanin) and liquid.

At step S130, the solid melanin is purified via washing with HCl and subsequent washing with deionized, distilled water. Washing with these solvents will remove any impurities (e.g., salt, polysaccharides, other fungal compounds) that are soluble in either water or HCl. In one or more embodiments, the washing is done by centrifugation or vacuum filtration. In at least one embodiment in which centrifugation is used, the melanin in the centrifuge tube is washed by adding either deionized, distilled water or HCl to the centrifuge tube and manually shaking to disperse the melanin pellet. The tube is then centrifuged to isolate the melanin pellet, and following centrifugation, the supernatant is removed. In one or more embodiments, the washing step is repeated until the supernatant is clear. In one or more embodiments, the solid melanin is first washed with HCl 1-3 times (via centrifugation or vacuum filtration), and then washed 1-2 times with deionized, distilled water (via centrifugation or vacuum filtration), which removes any remaining HCl. In at least one preferred embodiment, the solid melanin is first washed with HCl 3 times (via centrifugation or vacuum filtration), and then washed 2 times with deionized, distilled water (via centrifugation or vacuum filtration). In one or more embodiments, the HCl is diluted HCl. For example, the HCl can be less 0.5M HCl. In at least one embodiment, the diluted HCl is 0.15M HCl. In one or more embodiments, the washing step is done 3-4 times each with HCl and deionized, distilled water. In one or more embodiments, the washing steps can be done in an alternating fashion for HCl and deionized, distilled water (e.g., washing with HCl and then washing with deionized, distilled water such that the melanin is washed 3-4 times total. In at least one embodiment in which the washing is done by vacuum filtration, the melanin is placed on filter paper and is washed by pouring HCl or deionized, distilled water over it and filtering to pull the solvent through to the flask. In at least one embodiment, centrifugation and vacuum filtration can be used in combination to accomplish the purifying (washing) step of the melanin. For example, the solid melanin can undergo a first washing via centrifugation with HCl and subsequently a second washing via vacuum filtration with HCl, and similar alternation can occur for washing with deionized, distilled water.

At step S135, the solid melanin is dried. In one more embodiments, the solid melanin is dried by evaporation using a mineral oil bath, drying oven, or other method to remove remaining solvent from melanin pellet. In one or more embodiments, the solid melanin is heated at approximately 100° C. to evaporate any remaining solvent. In one or more embodiments, the solid melanin can be heated at a temperature higher than 100° C. In one or more embodiments, a laboratory oven or furnace be used to dry the solid melanin (and remove the remaining solvent) at a controlled temperature. In at least one embodiment, the heating is accomplished via irradiation with microwaves to remove water by boiling. Irradiation with microwaves quickly removes water and dries the product, and thus increases efficiency of the extraction process. In at least one embodiment, the solid melanin can be dried in a vacuum environment.

In an alternative embodiment, the solid melanin is washed and kept under vacuum. In this alternative embodiment, the solid melanin is then heated to evaporate any remaining water.

In at least one embodiment, after the melanin is dried, the dried melanin is combined with ethanol and re-dried at approximately 80° C.-100° C. to ensure complete solvent evaporation. Because melanin is hygroscopic, this additional drying step with ethanol helps to ensure the final solid melanin has as low a water content as possible.

Optionally, at step S140, once the solid melanin is dried, one or more melanin confirmation tests can be performed to ensure a high purity fungal melanin product. In at least one embodiment, the presence and/or purity of melanin can be evaluated by one or more of the following tests: the color of the solid melanin product, the solubility of the product in alkaline solutions, the solubility of the product in hot and cold concentrated acids, and the reaction of the product with a strong oxidizing agent. In one or more embodiments, all four of these tests are used to evaluate or confirm the presence and/or purity of melanin. The presence and/or purity of melanin can be evaluated via the above tests as follows: color of the solid melanin product is black-dark brown; the solid melanin product is soluble in alkaline solution; the solid melanin product is insoluble in water; the solid melanin product is insoluble in hot and cold concentrated acids; and the solid melanin product is bleached by a strong oxidizing agent. In at least one embodiment, the presence and/or purity of melanin can be evaluated via an Ultraviolet-visible (UV-VIS) spectroscopy confirmation test, which can be performed using a GENESYS Vis/UV-Vis Spectrophotometer as manufactured by ThermoFisher Scientific, for example. In one or more embodiments, fungal melanin is expected to show an absorption peak within the range of 220-350 nm. At step S145, the method ends.

An exemplary embodiment of the present method is show in the below example.

### Example 1

A sample of chaga mushroom was finely pulverized and combined with 1M NaOH in a round bottom flask in a ratio of approximately 0.06 g/mL. The resulting mixture was then refluxed for 2 hours at 100° C. in a mineral oil bath over a hot plate equipped with stirring. The mixture was then centrifuged at 4500 RCF for 15 minutes to separate a supernatant containing the melanin extract and a solid pellet. The supernatant (melanin extract) was pipetted out and the solid was discarded. The melanin extract was then acidified using 10% HCl to lower the pH in the liquid to approximately 2. The acidified melanin extract then underwent a vacuum filtration with a 0.22-micron filter paper to separate the solid (melanin) and liquid. The liquid was then discarded. Finally, the melanin was purified via washing (centrifugation) 3 times with 0.15M HCl and subsequently 2 times with deionized, distilled water to dilute any remaining HCl or remove other impurities.

### Example 2

In this example, melanin was extracted from a sample of chaga mushroom using the method described in Example 1. Additionally, melanin was extracted from a sample of three other species of fungi, Fomes fomentarius, Phellinus igniarius, and Ganoderma tsugae, using the same method as used for the extraction of melanin from the chaga mushroom in Example 1. The extraction methods for Fomes fomentarius, Phellinus igniarius, and Ganoderma tsugae resulted in 12%, 14%, and 3.5% yields of melanin, respectively ([mass of the extracted melanin substance/mass of the sample]×100). Surprisingly, the extraction method for the chaga mushroom resulted in approximately a 24% yield of melanin from the chaga mushroom sample. As such, the present method was shown to provide unexpected increased yield in melanin from chaga mushroom samples.

Overall, the methods of the present application provide an effective and efficient way for extracting melanin from fungal samples, and in particularly, fungal samples from Inonotus obliquus (chaga), Ganoderma tsugae (hemlock reishi), Fomes fomentarius (tinder fungus), or Phellinus igniarius (willow bracket). For example, as shown in the above example, the present methods produce nearly a 25% yield of melanin from a chaga mushroom sample. This is a substantial improvement over melanin yield from certain other fungal species, including Ganoderma tsugae as well as many previously known melanin extraction methods, such as the method of Prados-Rosales, Rafael et al., “Structural Characterization of Melanin Pigments from Commercial Preparations of the Edible Mushroom Auricularia auricula.” Journal of agricultural and food chemistry vol. 63, 33 (2015): 7326-32, which led to only a 10% yield of melanin. The present methods also do not include the use of enzymes or a dialysis step, making the present methods more efficient in terms of both costs and timing. Finally, the produced melanin extract of the present methods is useful for numerous downstream applications, such as topical sunscreens, facial and skin creams, metal chelation processes, and energy transduction processes, and viral and fungal and other medical research.

Although much of the foregoing description has been directed to methods for extracting melanin from a sample from Inonotus obliquus, Fomes fomentarius, or Phellinus igniarius, the methods disclosed herein can be similarly deployed and/or implemented in scenarios, situations, and settings far beyond the referenced scenarios. It should be further understood that any such implementation and/or deployment is within the scope of the composition and methods described herein.

It is to be further understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms ““including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings are shown accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure and equivalent structures and functions or steps.

What is claimed:

1. A method for extracting melanin from a fungal species, the method comprising:
drying and pulverizing a sample of a fungus, wherein the fungus is selected from the group consisting of Inonotus obliquus, Fomes fomentarius, Ganoderma tsugae, and Phellinus igniarius;
refluxing the sample in a sodium hydroxide (NaOH) solution to form a mixture;
separating the mixture into a liquid portion and a solid portion via centrifugation or vacuum filtration, wherein the centrifugation or vacuum filtration results in the liquid portion comprising melanin;
acidifying the separated liquid portion to a pH of approximately 2 or less than 2 with a hydrochloric acid (HCl) solution to precipitate melanin;
isolating the precipitated melanin via centrifugation or vacuum filtration;
purifying the melanin with at least one of HCl and deionized, distilled water; and
drying the purified melanin.

2. The method of claim 1, further comprising:
performing at least one melanin confirmation test on the purified melanin, wherein the at least one test is selected from the group consisting of: confirming that the purified melanin is black-dark brown, confirming that the purified melanin is soluble in an alkaline solution, confirming that the purified melanin is insoluble in hot and cold concentrated acids, and confirming that a reaction of the purified melanin with an oxidizing agent results in bleaching of the purified melanin.

3. The method of claim 1, wherein the NaOH solution has a molar concentration in a range of approximately 0.5M-2M.

4. The method of claim 3, wherein the NaOH solution is 1M NaOH and the pulverized sample is combined with the 1M NaOH in a container at a ratio of approximately 0.01-0.1 g/mL.

5. The method of claim 1, wherein the sample is refluxed for 1-5 hours at a temperature in the range of approximately 100° C.-103° C.

6. The method of claim 5, wherein the sample is refluxed for approximately 2 hours in a container and the container is immersed in a mineral oil bath maintained at approximately 100° C.

7. The method of claim 1, wherein the step of separating the mixture into a liquid portion and a solid portion via centrifugation or vacuum filtration comprises centrifuging the mixture at a relative centrifugal force (RCF) of approximately 4500-5000 for approximately 10-25 minutes.

8. The method of claim 1, wherein the step of separating the mixture into a liquid portion and a solid portion via centrifugation or vacuum filtration comprises vacuum filtrating the mixture using a vacuum pump and a 0.22-micron filtration paper.

9. The method of claim 1, wherein the HCl solution for acidifying the separated liquid portion is an approximately 10%-37% HCl solution.

10. The method of claim 1, wherein the step of isolating the precipitated melanin comprises centrifugation of the precipitated melanin at approximately 4500-5000 RCF for approximately 10-25 minutes or vacuum filtration of the precipitated melanin using a vacuum pump and a 0.22-micron filtration paper.

11. The method of claim 1, wherein the step of purifying the melanin comprises centrifuging or vacuum filtrating the melanin with HCl 3 times and subsequently centrifuging or vacuum filtrating the melanin with deionized distilled water 2 times, wherein the HCl is less than 0.5M HCl.

12. The method of claim 1, wherein the step of drying the melanin comprises:
heating the melanin at approximately 100° C. to evaporate any remaining water.

13. The method of claim 12, wherein the heating comprises irradiation with microwaves to remove water by boiling.

14. A method for extracting melanin from a Inonotus obliquus (chaga) mushroom, the method comprising:
drying and pulverizing a sample of chaga mushroom;
combining the sample with a 1M sodium hydroxide (NaOH) solution in a container in a ratio of approximately 0.01-0.1 g/mL to form a mixture;
refluxing the mixture for approximately 2 hours at approximately 100° C.;
separating the mixture via centrifugation at approximately 4500 RCF for approximately 10-25 minutes to form a liquid portion comprising melanin and a solid portion;
acidifying the separated liquid portion to a pH of approximately 2 or less than 2 with a hydrochloric acid (HCl) solution to precipitate melanin;
isolating the precipitated melanin via vacuum filtration;
purifying the melanin via centrifugation with at least one of HCl and deionized, distilled water; and
drying the purified melanin via irradiation with microwaves.

15. The method of claim 14, further comprising:
recycling the solid portion for further centrifugation to extract additional melanin.

16. The method of claim 14, wherein isolating the precipitated melanin comprises vacuum filtration using a vacuum pump and a 0.22-micron filtration paper.

17. The method of claim 14, wherein the step of purifying the melanin comprises centrifuging the melanin with HCl and subsequently centrifuging the melanin with deionized, distilled water.

18. A method for extracting melanin from an Inonotus obliquus (chaga) mushroom, the method comprising:
drying and pulverizing a sample of chaga mushroom;
combining the sample with a 1M sodium hydroxide (NaOH) solution in a container in a ratio of approximately 0.06 g/mL to form a mixture;
refluxing the mixture for approximately 2 hours at approximately 100° C.;
separating the mixture via vacuum filtration to form a liquid portion comprising melanin and a solid portion;
acidifying the separated liquid portion to a pH of approximately 2 or less than 2 with a hydrochloric acid (HCl) solution to precipitate melanin;
isolating the precipitated melanin via centrifugation at approximately 4500 RCF;
purifying the melanin via centrifugation with HCl and subsequent centrifugation with deionized, distilled water; and
drying the purified melanin via irradiation with microwaves.

19. The method of claim 18, wherein the precipitated melanin is vacuum filtrated using a vacuum pump and a 0.22-micron filtration paper.

20. The method of claim 18, wherein the step of isolating the precipitated melanin comprises centrifugation for approximately 10-25 minutes.
